SEQUENCE LISTING (1 sequence)

SEQ ID NO: 1 - protein, 4 aa, Artificial Sequence
  Synthetic sequence, tetra peptide from whey decompositions
GTWY

(19) United States
(12) Patent Application Publication
(10) Pub. No.: US 2021/0177930 A1
(43) Pub. Date: Jun. 17, 2021

(54) COMPOSITION FOR USE IN IMPROVING ATTENTION FUNCTION AND JUDGEMENT FUNCTION

(71) Applicant: KIRIN HOLDINGS KABUSHIKI KAISHA, Tokyo (JP)

(72) Inventors: Masahiro KITA, Tokyo (JP); Yasuhisa ANO, Tokyo (JP)

(73) Assignee: KIRIN HOLDINGS KABUSHIKI KAISHA, Tokyo (JP)

(21) Appl. No.: 17/259,778

(22) PCT Filed: Jul. 12, 2019
(86) PCT No.: PCT/JP2019/027622

§ 371 Date: Jan. 12, 2021

(30) Foreign Application Priority Data
Jul. 13, 2018 (JP) 2018-132907

(51) Int. Cl. A61K 38/07; A61P 25/28; A61K 38/05; A23L 33/19; A23L 33/18; A23L 33/00

(52) U.S. Cl. CPC A61K 38/07; A61P 25/28; A61K 38/05; A23V 2002/00; A23L 33/18; A23L 33/40; A23L 33/19

(57) ABSTRACT

An object of the present invention is to provide a composition for use in improving, maintaining and/or ameliorating an attention function and/or a judgement function. According to the present invention, there is provided a composition for use in improving, maintaining and/or ameliorating an attention function and/or a judgement function, comprising an enzymatic decomposition product of milk protein as an active ingredient. The enzymatic decomposition product of milk protein is preferably an enzymatic decomposition product of whey protein. The enzymatic decomposition product of milk protein preferably comprises a peptide having an amino acid sequence of GTWY (SEQ ID NO: 1) and a peptide having an amino acid sequence of WY.

### CROSS-REFERENCE TO RELATED APPLICATION

The present application enjoys the benefit of priority from the prior Japanese Patent Application No. 2018-132907 filed on Jul. 13, 2018, the entire disclosure of which is incorporated herein by reference.

### TECHNICAL FIELD

The present invention relates to a composition for use in improving an attention function and a judgement function.

### BACKGROUND ART

Maintenance, improvement, and amelioration of brain functions are required by a wide range of generations from the young to the elderly. The brain functions would deteriorate along with aging and fatigue, and attention and judgment constituting a cognitive function would also deteriorate. Especially, the deterioration in attention and judgement caused by fatigue is considered to lead to deterioration in productivity of intellectual work. Therefore, the improvement in productivity of intellectual work is expected by addressing the amelioration of an attention function, a judgment function and the like.

A search for substances that improve various brain functions has been made so far. In food materials, for example, chlorogenic acids are known to ameliorate an attention control function and an executive function (Patent Document 1).

### REFERENCE LIST

### Patent Documents

Patent Document 1: JP 2018-39797 A

### SUMMARY OF THE INVENTION

An object of the present invention is to provide a composition for use in improving, maintaining and/or ameliorating an attention function and/or a judgement function.

The present inventors conducted a verbal fluency test and the Stroop test on subjects who ingested an enzymatic decomposition product of milk protein to verify an attention function and a judgement function, and found improvements in the attention function and the judgement function. Further, the present inventors made stratified analysis of the subjects through the VAS inspection for the feeling of fatigue and the like to verify the attention function and judgement function of subjects prone to be fatigued, and found remarkable improvements in the attention function and the judgement function of these subjects. The present invention is based on these findings.

The present invention provides the following inventions.

[1] A composition for use in improving, maintaining and/or ameliorating an attention function and/or a judgement function (hereinafter sometimes referred to as “composition of the present invention”) and an agent for improving, maintaining and/or ameliorating an attention function and/or a judgement function (hereinafter sometimes referred to as “agent of the present invention”), each comprising an enzymatic decomposition product of milk protein as an active ingredient.

[2] The composition and agent according to [1], wherein the enzymatic decomposition product of milk protein is an enzymatic decomposition product of whey protein.

[3] The composition and agent according to [1] or [2], wherein the enzymatic decomposition product of milk protein comprises a peptide having an amino acid sequence of GTWY (SEQ ID NO: 1) and a peptide having an amino acid sequence of WY.

[4] The composition and agent according to any one of [1] to [3], which are each used to be provided to a subject who is prone to be fatigued by intellectual work for ingestion.

[5] The composition and agent according to any one of [1] to [4], wherein the intellectual work is work which requires attention and/or concentration.

[6] The composition and agent according to any one of [1] to [5], which are each used to be provided to a subject who is fatigued or a subject who is prone to be fatigued in daily life for ingestion.

[7] The composition and agent according to any one of [1] to [6], which each provide the enzymatic decomposition product of milk protein in the amount of 0.01 to 100 g (in terms of solid content) for a human per day for ingestion.

[8] The composition and agent according to any one of [1] to [7], which are each in the form of a food.

[9] A method for improving, maintaining or ameliorating an attention function and/or a judgement function, comprising feeding or administering an effective amount of an enzymatic decomposition product of milk protein or a composition comprising the enzymatic decomposition product of milk protein to a subject in need thereof.

[10] Use of an enzymatic decomposition product of milk protein or a composition comprising the enzymatic decomposition product of milk protein, for the manufacture of an agent for improving, maintaining and/or ameliorating an attention function and/or a judgement function, as an agent for improving, maintaining and/or ameliorating an attention function and/or a judgement function, or for improving, maintaining and/or ameliorating an attention function and/or a judgement function.

[11] An enzymatic decomposition product of milk protein for use in improving, maintaining and/or ameliorating an attention function and/or a judgement function.

The decomposition product of milk protein, which is the active ingredient of the composition and agent of the present invention, is an enzymatic decomposition product of milk protein including whey protein which has been eaten for a long time. Thus, the composition and agent of the present invention are advantageous in that they can be used as functional foods which exhibit the function of improving, maintaining and/or ameliorating an attention function and/or a judgement function, and can also be used as safe functional foods for mammals including humans.

### DETAILED DESCRIPTION OF THE INVENTION

The enzymatic decomposition product of milk protein, which is the active ingredient of the composition and agent of the present invention, (hereinafter sometimes referred to as “decomposition product of milk protein of the present invention”) is not particularly limited as long as it is an enzymatic decomposition product of milk protein. The milk protein can be derived from whole milk, powdered milk, casein or whey, and is preferably derived from whey. The “whey” is also referred to as milk serum, serum lactis or whey, and means an aqueous solution obtained by removing a milk fat component, casein and the like from milk. The whey is composed of β-lactoglobulin, α-lactalbumin, serum albumin, immunoglobulin and the like. The whey used in the present invention may be derived from any animal or plant, but is preferably cow milk-derived whey. The decomposition product of milk protein of the present invention is preferably an enzymatic decomposition product of whey protein (hereinafter sometimes referred to simply as “decomposition product of whey”).

The composition and agent of the present invention can each comprise the decomposition product of milk protein of the present invention alone, or can comprise the decomposition product of milk protein as a mixture with any other component. The content (in terms of solid content) of the decomposition product of milk protein of the present invention in each of the composition and agent of the present invention can be optionally determined depending, for example, on the purpose, intended use, form, dosage form, symptom, age and the like, and can be set to, for example, 0.001% to 99% by mass (preferably, 0.01% to 95% by mass) based on the total amount although the present invention is not limited thereto. In the present invention, the agent of the present invention can consist of the decomposition product of milk protein of the present invention, and the composition of the present invention can comprise the decomposition product of milk protein of the present invention and any other component.

As the decomposition product of milk protein (especially, decomposition product of whey) of the present invention, a decomposition product of milk protein comprising a peptide having an amino acid sequence of GTWY (SEQ ID NO: 1) and a peptide having an amino acid sequence of WY (hereinafter sometimes referred to collectively as “peptide of the present invention”) can be used. The phrase “peptide having an amino acid sequence” means a peptide of which sequence is specified by the amino acid sequence.

The content (in terms of solid content) of the tetrapeptide GTWY in the decomposition product of milk protein (especially, decomposition product of whey) of the present invention is, for example, 0.01% to 1% by mass (preferably, 0.05% to 0.5% by mass), and the content (in terms of solid content) of the dipeptide WY is, for example, 0.005% to 0.5% by mass (preferably, 0.01% to 0.1% by mass).

The content (in terms of solid content) of the peptide of the present invention in each of the composition and agent of the present invention can be optionally determined depending, for example, on the purpose, intended use, form, dosage form, symptom, body weight and the like, and can be set to, for example, 0.00001% to 50% by mass (preferably, 0.0001% to 10% by mass) based on the total amount although the present invention is not limited thereto.

The method for manufacturing the decomposition product of milk protein of the present invention (especially, decomposition product of milk protein containing the peptide of the present invention) is known, and the decomposition product of milk protein can be manufactured according to WO 2017/086303. Alternatively, a commercial decomposition product of whey (for example, HW-3 (manufactured by MEGMILK SNOW BRAND Co., Ltd.)) may be used as the decomposition product of milk protein of the present invention.

The decomposition product of milk protein containing the peptide of the present invention can be manufactured, for example, by causing an enzyme preparation comprising a proteolytic enzyme to act on a raw material protein.

When whey protein is used as the raw material, the concentration of the whey protein subjected to the enzymatic reaction is not limited as long as the protein can be dissolved. However, from the viewpoint of suppressing gelation and agglomeration and saving a concentrating step, the concentration is preferably 1 to 30 w/v %, more preferably 1 to 20 w/v %, further preferably 5 to 15 w/v %.

When the raw material protein is an aqueous solution such as milk serum or serum lactis, the raw material protein can be subjected to the enzymatic reaction as it is or in a concentrated or diluted state, and, if necessary, the pH or the like can be adjusted. When the raw material protein is a solid such as powder, it may be dissolved in any aqueous solvent as long as the enzymatic reaction progresses, but, in consideration of its use as a food, it is preferably dissolved in water or a food additive grade buffer solution. A buffer solution is preferably used in order to prevent the pH of the reaction solution from changing due to the amino acids generated by the enzymatic reaction. The type of the buffer solution is arbitrary, and can be determined in consideration of the subsequent use, flavor, taste and mineral amount. The buffer solution preferably has a composition which allows the pH of the reaction solution to be maintained in the range of 4 to 9, preferably 5 to 8, more preferably 7 to 8. Most preferably, the buffer solution is a potassium phosphate buffer solution. The buffer solution may have any concentration as long as the buffer effect can be obtained, but, in consideration of the flavor, taste and mineral amount, the concentration can be set to 0.01 to 0.5 M, preferably 0.05 to 0.2 M, more preferably about 0.1 M.

Any enzyme preparation can be used as long as it comprises a proteolytic enzyme. However, the enzyme preparation is preferably an enzyme preparation comprising neutral protease, and one or more thereof can be used in combination. As the enzyme preparation, those derived from a microorganism such as Bacillus subtilis, Aspergillus oryzae or Aspergillus melleus can be used. Among these, an enzyme preparation derived from Aspergillus oryzae and an enzyme preparation derived from Aspergillus melleus are preferred, and an enzyme preparation derived from Aspergillus melleus is more preferred.

In the present invention, a commercial enzyme preparation can be used. For example, the enzyme preparation is available from Amano Enzyme Inc., SHINNIHON CHEMICALS Corporation, DSM, Danisco A/S, Novozymes A/S, HBI Enzymes Inc., and the like. The amount of the enzyme preparation to be added is arbitrary, but, in consideration of an appropriate hydrolysis reaction rate and cost, can be set to, for example 0.01 to 5 w/v %, preferably 0.05 to 4 w/v %, more preferably 0.1 to 0.5 w/v %.

The enzymatic reaction temperature and enzymatic reaction time can be set so that the raw material protein is sufficiently hydrolyzed and that the quality of the enzymatic decomposition product is maintained. Specifically, the enzymatic reaction temperature can be set to, for example, 30 to 70° C., preferably 40 to 70° C., more preferably 45 to 65° C. The enzymatic reaction time can be set to 1 to 12 hours, preferably 2 to 10 hours, more preferably 4 to 5 hours. It is possible to appropriately adjust the reaction temperature and reaction time while checking the amount of the peptide of the present invention produced.

It is also possible to carry out the enzymatic reaction while increasing the temperature. For example, it is possible to carry out the enzymatic reaction while increasing the temperature from 30° C. up to 75° C. over 4 to 10 hours. It is possible to carry out the enzymatic reaction while increasing the temperature preferably from 35° C. up to 75° C. over 5 to 8 hours, more preferably from 35° C. up to 75° C. over 6 to 8 hours. The temperature increase speed is arbitrary, but, preferably, the temperature is retained within the range of from 45° C. up to 55° C. for a longer time (5 to 7 hours), then rapidly increased up to 60° C. and then retained within the range of from 60° C. to 75° C. for a longer time (for example, 1 to 3 hours). The most preferable method involves adding the enzyme at 50° C., retaining it for 5 to 7 hours, then increasing the temperature at an arbitrary rate, and retaining it at a target temperature of 60 to 65° C. or 65 to 75° C. for 1 to 3 hours.

In the reaction, the reaction solution is preferably stirred from the viewpoint of reaction efficiency. The solution stirring rate is preferably higher for increasing frequency of contact of the substrate with the enzyme, but, if the solution stirring rate is too high, the reaction solution may splash. Therefore, the solution stirring rate can be set to, for example 100 to 500 rpm, preferably 200 to 400 rpm, more preferably about 250 rpm.

Once the desired peptide is obtained, the reaction solution is preferably subjected to a step of terminating the enzymatic reaction. In the termination step of the enzymatic reaction, a method of increasing the temperature of the reaction solution or adding a chelating agent to change the chemical structure of the enzyme or a method of removing the enzyme through membrane treatment can be adopted. A preferred method is deactivation treatment at a high temperature. This treatment can be carried out by retaining the reaction solution at 80 to 90° C. for 5 to 30 minutes, preferably at 80 to 90° C. for 20 to 30 minutes. Further, when the temperature becomes high in a concentration step which will be described later, this step can serve also as the concentration step.

The reaction solution (decomposition product of milk protein) that has undergone the enzymatic reaction step and the termination step of the enzymatic reaction may be further subjected to a sterilization step. Examples of the sterilization step include a membrane treatment step or a high-temperature sterilization step which will be described later. The high-temperature sterilization step can serve also as the enzymatic reaction termination step, which is advantageous in simplification of the manufacturing process.

The reaction solution (decomposition product of milk protein) that has undergone the enzymatic reaction step and the termination step of the enzymatic reaction may be further subjected to a purification step. Examples of the purification step include a membrane treatment step, and preferable membrane treatment is ultrafiltration treatment. The molecular weight cutoff in the ultrafiltration treatment is preferably 3 to 100 kDa, more preferably 5 to 50 kDa. Carrying out the purification step is advantageous in that the flavor of the peptide composition can be ameliorated as compared with the case where the purification step is not carried out. The purification step can serve also as the termination step of the enzymatic reaction and the sterilization step, which is advantageous also in simplification of the manufacturing process.

The reaction solution (decomposition product of milk protein) that has undergone the enzymatic reaction step and the termination step of the enzymatic reaction may be further subjected to the concentration step, from the viewpoint of storage and transportation. Any method can be selected as the concentration step, but the method is preferably a method of concentration under reduced pressure and methods of concentration by freeze drying, spray drying and membrane treatment (for example, a method using a reverse osmosis membrane), more preferably freeze-drying and spray drying. Spray drying is particularly preferred from the viewpoint of efficiently carrying out concentration in a large amount.

The content of the peptide of the present invention can be measured by liquid chromatography tandem mass spectrometry (LC/MS/MS). Those skilled in the art can easily set the conditions for LC/MS/MS, and, needless to say, a peptide having a suitable purity for LC/MS/MS measurement is used as the standard peptide at the time of measurement. For example, AQUA Peptide manufactured by SIGMA ALDRICH can be used.

The composition and agent of the present invention are each used to improve, maintain and/or ameliorate an attention function. The “attention function” means a function of consciously or intentionally focusing mental energy on one object or one component of a complex experience and eliminating other emotional or thinking contents, and is defined as mainly including a concentration function/selection function (function of focusing on a certain stimulus), a maintenance function (function of maintaining the intensity of attention for a certain period of time), and a control function (function of selecting information to attain the purpose or paying attention to two stimuli at the same time) (Clinical Assessment for Attention⋅Clinical Assessment for Spontaneity, edited by Japan Society for Higher Brain Dysfunction, Shinkoh Igaku Shuppansha Co., Ltd. (2008)). In other words, the “attention function” can also be said to be a function of turning consciousness only to a stimulus necessary to attain the goal, or allocating consciousness to a plurality of stimuli and maintaining it for a certain period of time.

The composition and agent of the present invention are each used to improve, maintain and/or ameliorate a judgment function. The “judgement function” means a higher-order function of transforming behavior and optimizing response to an unfamiliar situation, and has the same meaning as the “executive function.” The “judgement function” is defined as being important when making plans for the future, voluntarily transforming actions, and resisting temptation (Sam J. G et al., Current Biology, 18(3), 110-114 (2008)). The major elements of the “judgement function” are updating of information, shifting of task rules (flexibility of thinking), and suppression of reactions (Miyake A. et al., Cognitive Psychology, 41(1), 49-100 (2000)). The “judgement function” is also known to include “working memory,” “flexibility of thinking” and “planning” (JOHN R HODGES, “Cognitive Assessment for Clinicians,” Shinkoh Igaku Shuppansha Co., Ltd., 2011, pp. 24-26, ISBN978-4-88002-4; and Wiebe SA. et al, Dev Psychol. 2008 March; 44(2): 575-87). In other words, the “judgement function” can also be said to be a function of flexibly judging things and suppressing unnecessary actions so that humans perform activities along their plan.

The composition and agent of the present invention can be each provided to a subject who is prone to be fatigued by intellectual work for ingestion. The intellectual work may be intellectual work which requires attention and/or concentration. The composition and agent of the present invention can be each provided to a subject who is fatigued or prone to be fatigued in daily life for ingestion.

The attention function and judgment function to be improved, maintained and/or ameliorated by the composition and agent of the present invention are distinguished from the brain functions impaired by cranial nerve diseases. Specifically, the composition and agent of the present invention can be each provided for ingestion to a subject who does not suffer from cranial nerve diseases such as dementia, schizophrenia, Alzheimer's disease, Parkinson's disease, amyotrophic lateral sclerosis and the like, and a subject who is not diagnosed as suffering from any of the cranial nerve diseases listed above or being at a risk thereof. Briefly, the composition and agent of the present invention can be each used for a healthy subject.

In the present invention, the “improvement of a function” includes, for example, achieving a higher level of the function than the current state. Further, the “maintenance of a function” includes, for example, preventing deterioration in the function. Further, the “amelioration of a function” includes, for example, recovering the once deteriorated function and a symptom having a sign of deterioration. In the present invention, the meaning of the “improvement, maintenance and/or amelioration of a function” includes enhancement of the function and suppression of deterioration in the function.

The composition and agent of the present invention can be each provided in the form of a pharmaceutical product (for example, pharmaceutical composition), a quasi-drug, a food, a feed (including a pet food) or the like, and can be implemented according to the following descriptions.

The composition and agent of the present invention can be each administered orally to a human and a non-human animal. Examples of oral formulations include granules, powders, tablets (including sugar-coated tablets), pills, capsules, syrups, emulsions, and suspensions. These formulations can be formulated using pharmaceutically acceptable carriers by techniques commonly used in the art. Pharmaceutically acceptable carriers include excipients, binders, diluents, additives, flavoring agents, buffers, thickeners, colorants, stabilizers, emulsifiers, dispersants, suspending agents, preservatives and the like.

When the decomposition product of milk protein of the present invention is provided as a food, it can be provided as a food as it is or provided in a state of being contained in a food. When the decomposition product of milk protein of the present invention is provided as a food, for example, an enzymatic decomposition product of milk protein such as a decomposition product of whey can be provided as a food as it is or provided in a state of being contained in a food. The food thus provided is a food containing an effective amount of the decomposition product of milk protein of the present invention. The phrase “containing an effective amount” of the decomposition product of milk protein of the present invention refers to a content of the decomposition product of milk protein to be ingested within a range as will be described later, when a normally-eaten amount of individual foods is ingested. The meaning of the “food,” as used herein, includes health foods, functional foods, nutritional supplements, foods with health claims (such as foods for specified health uses, foods with nutrient function claims, and foods labeled with functions), foods for special dietary uses (such as foods for infants, foods for expectant and nursing mothers and foods for sick persons) and supplements. When the decomposition product of milk protein of the present invention is provided to an animal other than a human for ingestion, needless to say, the food referred to herein is used as a feed.

The decomposition product of milk protein of the present invention has an effect for improving, maintaining and ameliorating an attention function and a judgement function as described above, and thus can be contained in foods ingested daily or provided as supplements. Briefly, the composition and agent of the present invention can be each provided in the form of a food. In this case, the composition and agent of the present invention can be each provided in the form of a unit package in which the ingestion amount per meal is predetermined. Examples of the unit package form per meal include forms which define a constant amount using a pack, a package, a can, a bottle and the like. To exert various actions of the composition and agent of the present invention better, the ingestion amount per meal can be determined according to the daily ingestion amount for the attention function and/or judgement function of the present invention which will be described later. The food of the present invention may be provided in the form of a package on which an explanation about the ingestion amount is given, or provided together with a document or the like which explains the ingestion amount.

The predetermined ingestion amount per meal in the unit package form may be either the effective daily ingestion amount or an ingestion amount obtained by dividing the effective daily ingestion amount into two or more (preferably two or three) portions. Thus, the unit package form of the composition and agent of the present invention can contain the decomposition product of milk protein of the present invention in the daily ingestion amount for a human which will be described later, or can contain the decomposition product of milk protein of the present invention in an amount half or one sixth of the daily ingestion amount for a human which will be described later. For convenience of ingestion, the composition and agent of the present invention are each preferably provided in “the form of a unit package per meal” in which the ingestion amount per meal is the effective daily ingestion amount.

The form of the “food” is not particularly limited, and the food may be provided, for example, in a beverage form, in a semi-liquid or gelled form, or in a solid or powder form. Examples of the “supplement” include tablets manufactured by adding an excipient, a binder and the like to the decomposition product of milk protein of the present invention, kneading them together and then tableting the kneaded product, and capsule agents in which the decomposition product of milk protein of the present invention is encapsulated in a capsule and the like.

The food provided in the present invention is not particularly limited as long as it contains the decomposition product of milk protein of the present invention, and examples thereof can include non-alcoholic beverages such as refreshing drinks, carbonated drinks, drinks containing fruit juice, drinks containing vegetable juice, drinks containing fruit juice and vegetable juice, animal milk such as cow milk and the like, soybean milk, milk beverages, lactic acid bacterium beverages, drink-type yogurt, drink-type and stick-type jellies, coffee, cocoa, tea drinks, nutritional drinks, energy drinks, sports drinks, mineral water, flavored water, non-alcohol beer-taste beverages and the like; carbohydrate-containing foods and beverages such as rice, noodles, bread, pasta and the like; dairy products such as cheese, hard-type or soft-type yogurt, fresh cream composed of animal milk and other oil and fat raw materials and ice cream; various confectioneries such as Western-style confectioneries including cookies, cakes, chocolate and the like, Japanese-style confectioneries including buns with a bean-jam filling. sweet jellies of adzuki beans and the like, tablet confectioneries (refreshing confectioneries) including soda pop-flavored sweets, candies, gums, chilled sweets and frozen sweets including jellies, puddings and the like, snacks and the like; alcoholic beverages such as whiskey, bourbon, spirit, liqueur, wine, fruit wine, sake (Japanese rice wine), Chinese liquor, shochu (Japanese distilled spirit), beer, non-alcohol beer having an alcohol content of 1% or less, low-malt beer, other miscellaneous liquors, shochu highball and the like; processed food products such as processed products in which eggs are used, processed products of fish and meat (including giblets such as lever) (including rare delicacy), soup and the like including miso soup and the like, flavorings such as miso, soy sauce, rice seasoning and other seasonings, and liquid diets such as high density liquid diets and the like. It should be noted that mineral water includes both of effervescent mineral water and non-effervescent mineral water. The food which is provided in the present invention contains both a food manufacturing raw material and a food additive.

Tea drinks include all of fermented tea, semi-fermented tea and unfermented tea, and examples thereof include black tea, green tea, barley tea, genmai cha (coarse green tea mixed with roasted brown rice), sencha (ordinary green tea), gyokuro cha (refined green tea), hoji cha (roasted green tea), oolong tea, turmeric herbal tea, pu-erth tea, rooibos tea, rose tea, chrysanthemum tea, ginkgo leaf tea and herb tea (such as mint tea and jasmine tea).

Examples of fruits used in drinks containing fruit juice and drinks containing fruit juice and vegetable juice include apple, orange, grape, banana, pear, peach, mango, acai, blueberry and plum. Examples of vegetables used in drinks containing vegetable juice and drinks containing fruit juice and vegetable juice include tomato, carrot, celery, pumpkin, cucumber and watermelon.

The decomposition product of milk protein of the present invention is an ingredient included, for example, in decomposition products of milk protein which have been ingested as foods by humans for a long time, and thus is less toxic and can be safely used for mammals in need thereof (such as humans, mice, rats, rabbits, dogs, cats, cows, horses, pigs, monkeys, dolphins and sea lions). The ingestion amounts or doses of the decomposition product of milk protein of the present invention and the peptide of the present invention can be determined depending, for example, on the sex, age, body weight and symptoms of a recipient, administration time, dosage form, administration route and drug to be combined. The daily ingestion amount and dose (in terms of solid content) for an adult of the decomposition product of milk protein of the present invention which are intended to improve, maintain and ameliorate an attention function and a judgment function are, for example, 0.01 to 100 g (preferably, 0.1 to 10 g). The daily ingestion amount and dose (in terms of solid content) for an adult of the peptide of the present invention which are intended to improve, maintain and ameliorate an attention function and a judgment function are, for example, 0.01 to 100 mg (preferably, 0.1 to 10 mg).

For example, it is desirable to initiate ingestion of the decomposition product of milk protein of the present invention before the time when, for example, the improvement of an attention function and a judgment function is expected, and the initiation timing can be set to 3 days before (preferably 5 days before, more preferably 10 days before) the expectation time. For example, it is desirable to ingest the decomposition product of milk protein of the present invention for as long a period of time as possible before the time when, for example, the improvement of an attention function and a judgment function is expected. The termination timing of the ingestion can be determined from this viewpoint, and set to, for example, 3 days before, 2 days before, 1 day before or the day of the expectation date.

The ingestion period of the decomposition product of milk protein of the present invention can be at least 2 days (preferably 3 days, more preferably 5 days, particularly preferably 10 days) for the daily ingestion amount described above. The ingestion interval of the decomposition product of milk protein of the present invention can be once every 3 days, once every 2 days, or once daily for the daily ingestion amount described above.

The above-described ingestion amount, ingestion timing, ingestion period and ingestion interval for the decomposition product of milk protein of the present invention and the peptide of the present invention are applicable when the decomposition product of milk protein of the present invention and the peptide of the present invention are used for both non-therapeutic and therapeutic purposes, and the “ingestion” (feeding) can be read as “administration” in the case of therapeutic purposes.

The composition and agent as well as food of the present invention may be labeled as having an effect for improving, maintaining and/or ameliorating an attention function and/or a judgement function. In this case, the label for the composition and agent as well as food of the present invention may contain some or all of the following indications for better understanding of consumers. Needless to say, the meaning of the phrase “improving, maintaining and/or ameliorating an attention function and/or a judgement function,” as used herein, includes the following indications:
for those who want to enhance attention at work;
for those who want to enhance concentration at work;
for those who are liable to be inattentive;
for those who often make careless mistakes;
for those who are easily distracted;
for those who cannot do multiple tasks at the same time;
for those who cannot continue to concentrate;
for those who are easily confused;
for those who forget small things quickly;
for those who are not good at flexible thinking;
for those who are not good at making and proceeding with plans;
for those who are prone to be fatigued by brainwork;
for those who easily feel brain fatigue;
for those who are prone to be fatigued in daily life;
for those who want to live an intelligent and positive life;
for those who want to keep an intelligent brain;
for those who want to keep a clear head;
function of maintaining attention and concentration for those who are aware of brain weakness and easily feel brain fatigue;
function of maintaining attention and concentration for those who are aware of brain weakness and easily feel fatigue by work requiring attention and concentration; and
function of maintaining attention and concentration for those who are aware of brain weakness and easily feel fatigue in daily life.

According to another aspect of the present invention, there is provided a method for improving, maintaining or ameliorating an attention function and/or a judgement function, comprising feeding or administering an effective amount of the enzymatic decomposition product of milk protein of the present invention or a composition comprising the enzymatic decomposition product of milk protein to a subject in need thereof. The method of the present invention can be carried out according to the descriptions about the composition and agent according to the present invention.

According to still another aspect of the present invention, there is provided use of the enzymatic decomposition product of milk protein of the present invention for the manufacture of an agent for improving, maintaining and/or ameliorating an attention function and/or a judgement function. According to still another aspect of the present invention, there is provided use of the enzymatic decomposition product of milk protein of the present invention as an agent for improving, maintaining and/or ameliorating an attention function and/or a judgement function. According to still another aspect of the present invention, there is provided use of the enzymatic decomposition product of milk protein of the present invention for improving, maintaining and/or ameliorating an attention function and/or a judgement function. The uses of the present invention can be carried out according to the descriptions about the composition and agent of the present invention.

According to yet another aspect of the present invention, there is provided an enzymatic decomposition product of milk protein for use in improving, maintaining and/or ameliorating an attention function and/or a judgement function. The enzymatic decomposition product of milk protein of the present invention can be carried out according to the descriptions about the composition and agent according to the present invention.

The method of the present invention and the use of the present invention may be use in mammals including humans and are intended to involve both of therapeutic use and non-therapeutic use. The “non-therapeutic,” as used herein, means elimination of operating, treating or diagnosing activities to a human (i.e., medical activities to a human), and specifically means elimination of a method of performing operation or treatment of, or diagnosis involving, a human by a doctor or a person who receives an instruction from the doctor.

### EXAMPLES

Hereinafter, the present invention will be described in more detail by way of the following examples, but is not limited thereto.

### Example 1: Preparation of Tablet Containing Decomposition Product of Whey and Measurement of Contents of Tetrapeptide GTWY and Dipeptide WY

### (1) Preparation of Tablet

A decomposition product of whey (HW-3 manufactured by MEGMILK SNOW BRAND Co., Ltd.), an excipient and a binder were mixed and kneaded, and the mixture was then tableted to manufacture a tablet containing the decomposition product of whey (210 mg/tablet). The content of the decomposition product of whey in one tablet was 168 mg. The decomposition product of whey (HW-3) is a product obtained by causing an enzyme preparation containing a proteolytic enzyme to act on whey protein, then performing membrane treatment to remove an undecomposed product, and drying the resultant product, and contains a tetrapeptide GTWY and a dipeptide WY as will be described later.

### (2) Preparation of Analytical Sample

One hundred (100) tablets (about 21 g) prepared in the above item (1) were thoroughly ground in a mortar, 20 mg of the ground product was weighed, and 1 mL of sterile water was added to suspend it thoroughly. The suspension was centrifuged (15,000 rpm, room temperature, 3 minutes) to obtain supernatant, and 500 μL of the supernatant was filtered through an ultrafiltration filter (10 kDa). The filtrate was diluted 1000 times to prepare a measurement sample.

### (3) Analysis Method

The concentrations of the tetrapeptide GTWY and the dipeptide WY in the measurement sample obtained in the above item (2) were determined by the LC/MS/MS method under the following analysis conditions. The GTWY concentration of the measurement sample was calculated by a calibration curve method using AQUA Peptide (manufactured by Sigma-Aldrich Co. LLC) as the standard sample.

### <Analysis Conditions>

Mass spectrometer: 4000Q TRAP (manufactured by AB Sciex Pte Ltd.) HPLC device: Agilent 1200 Series (manufactured by Agilent Technologies, Inc.) Column: TSK gel ODS-100V 3 μm 2.0 mm I.D.X 150 mm (manufactured by Tosoh Corporation)

Column temperature: 70° C.
Mobile phase A: 0.1% aqueous formic acid solution
Mobile phase B: 0.1% formic acid-acetonitrile solution
Gradient conditions: The gradient conditions indicated in Table 1 were applied.

TABLE 1
Gradient condition

| Total time | Ratio of mobile phase A | Ratio of mobile phase B |
|---|---|---|
| (min) | (volume %) | (volume %) |
| 0.00 | 100 | 0 |
| 10.00 | 80 | 20 |
| 30.00 | 20 | 80 |
| 30.01 | 0 | 100 |
| 40.00 | 0 | 100 |

Flow rate: 0.2 mL/min.
Sample injection volume: 2 μL
Ionization method: ESI (positive ion detection mode)

### Curtain gas: 40 psi

### Nebulizer gas: 50 psi

### Drying gas: 80 psi

Drying gas temperature: 600° C.
Collision gas: nitrogen
Ionizing voltage: 5000 V

### <Analysis Conditions for Tetrapeptide GTWY>

Set mass number (m/z)/collision energy (eV): 526.4→159.2/47, 526.4→368.3/23
DP voltage (V): 36

### <Analysis Conditions for Dipeptide WY>

Set mass number (m/z)/collision energy (eV): 368.2→351.1/19, 368.2→159.2/33
DP voltage (V): 51

### (4) Analysis Results

One tablet (210 mg) containing the decomposition product of whey was confirmed to contain 0.27 mg (0.129% by mass) of the tetrapeptide GTWY and 0.11 mg (0.052% by mass) of the dipeptide WY.

### Example 2: Verification of Attention Function Enhancing Effect and Judgement Function Enhancing Effect of Decomposition Product of Whey

### (1) Outline of Test

In Example 2, a test for verifying the effects of the decomposition product of whey on the attention function and the judgement function was conducted. This test was a placebo-controlled, randomized, double blind, parallel-group comparative test. The test period was set to 12 weeks, and a test food or a control food was provided for ingestion during the test period. Specifically, the “tablet containing the decomposition product of whey” and the “tablet free of the decomposition product of whey” were ingested as the test food and the control food, respectively, by healthy men and women of 45 years old and over and 64 years old and under, without dementia, who were aware of their own frequent forgetfulness and many mistakes, to confirm the effects of the decomposition product of whey on the attention function and the judgement function.

### (2) Subject

In a preliminary examination, subjects determined to be healthy by a doctor were randomly allocated to a test food group (50 subjects) and a control food group (51 subjects). The number of subjects as the target for analysis was 48 (men: 17 and women: 31) for the test food group and 50 (men: 17 and women: 33) for the control food group, and the age (average value±standard deviation) of subjects as the target for analysis was 52.3±4.3 years old for the test food group and 51.8±5.2 years old for the control food group. During the test period, the subjects were made to continue life similar to that before the test period.

### (3) Test Food

During the test period (12 weeks), 6 tablets of the test food and 6 tablets of the control food were ingested by subjects of the test food group and the control food group, respectively, once daily together with water or lukewarm water. As the test food, the “tablet containing the decomposition product of whey” manufactured in Example 1(1) (containing 168 mg of the decomposition product of whey in one tablet (210 mg)) was used. As the control food, the tablet free of the decomposition product of whey, manufactured by the same method as in Example 1(1) except that 168 mg of maltodextrin was blended in place of the decomposition product of whey, was used.

### (4) Measurement

### A. Measurement Items

The measurement items were the following four items.
Verbal fluency test
Stroop test
Visual Analogue Scale inspection for the feeling of fatigue (sometimes referred to as “VAS inspection for the feeling of fatigue” herein)
Profile of Mood States 2nd Edition (sometimes referred to as “POMS2 inspection” herein)

### B. Measurement Timing

Each of the verbal fluency test, Stroop test and POMS2 inspection was conducted once at each of visits of the subjects before the initiation of ingestion of the test food, at Week 6 after the initiation of ingestion, and at Week 12 after the initiation of ingestion, as indicated in Table 2. Note that, on the day of the test, the food was ingested 30 minutes before the beginning of the test. In addition, the VAS inspection for the feeling of fatigue was conducted once before and after the implementation of the verbal fluency test and the Stroop test (twice, in total) at each of the visits described above.

TABLE 2
Outline of measurement test schedule

| | Before | Week 6 after | Week 12 after |
|---|---|---|---|
| | initiation | initiation | initiation |
| | of ingestion | of ingestion | of ingestion |
| Verbal fluency test | Applicable | Applicable | Applicable |
| Stroop test | Applicable | Applicable | Applicable |
| VAS inspection for | Applicable | Applicable | Applicable |
| feeling of fatigue | (twice) | (twice) | (twice) |
| POMS2 inspection | Applicable | Applicable | Applicable |

C. Measurement method

### (i) Verbal Fluency Test

The subjects were made to verbally list as many words as possible falling under the two items “words starting with ‘a’” and “animal names.” The number of words for each of the items listed in one minute was evaluated. The ability to search for appropriate words across their own vocabulary is required to perform the verbal fluency test, and is used as an index of the judgement function (executive function) (JOHN R HODGES, “Cognitive Assessment for Clinicians,” Shinkoh Igaku Shuppansha Co., Ltd., pp. 135-137, 2011).

### (ii) Stroop Test

The subjects were made to read aloud “a meaning of a word composed of characters in a character string printed in a color different from the meaning of the word composed of the characters” or “a color of characters in a character string printed in a color different from the meaning of a word composed of the characters” as quickly as possible. The number of errors when the subjects read aloud such meanings or colors was evaluated. The ability to suppress typical erroneous reactions is required to perform the Stroop task, and is used as an index of the attention function and judgement function (executive function) (Toshio YAMAUCHI, Haruo KASHIMA and Shozo AOKI, “Handbook for Evaluation of Mental/Psychological Functions,” Nakayama Shoten Co., Ltd., pp. 133-135, 2015).

(iii) VAS Inspection for Feeling of Fatigue

The VAS inspection for the feeling of fatigue was conducted according to the VAS inspection method for the feeling of fatigue established by the Japanese Society of Fatigue Science. The subjects were made to put a “x” mark to represent the current feeling of fatigue felt by the subjects on a 100-mm straight line, with the “best feeling such that the subjects had no feeling of fatigue” set at the left end and the “worst feeling such that the subjects were so fatigued that they could not do anything” set at the right end. The distance from the left end to the “x” mark was evaluated.

### (iv) POMS2 Inspection

The subjects were made to answer a total of 35 questions about the mood states felt by the subjects in the past week. For each of the items, the answers were calculated based on the seven scales: “Anger-Hostility”, “Confusion-Bewilderment”, “Depression-Dejection”, “Fatigue-Inertia”, “Tension-Anxiety”, “Vigor-Activity”, and “Friendliness,” to evaluate the mood states in the past week. The scores on each of the scales were evaluated as T scores (values converted so as to approximate a normal distribution with a generation mean of 50 and a standard deviation of 10).

### (5) Evaluation and Analysis

Analysis of all the target subjects (overall analysis) and analysis of only the target subjects with a relatively high degree of fatigue evaluated by the VAS inspection for the feeling of fatigue and the POMS2 inspection (stratified analysis) were made.

### A. Method of Evaluating Numerical Value

In the verbal fluency test and the Stroop test, the actually measured values at each time point of measurement were obtained, and the values obtained by subtracting the corresponding actually measured values before the initiation of ingestion from the actually measured values after the initiation of ingestion were defined as amounts of change. The actually measured values and amounts of change for the two groups were evaluated using the two-sample t-test. For the changes from the values before the initiation of ingestion for the two groups, the actually measured values were evaluated using the one-sample t-test.

### B. Method of Stratified Analysis

(i) Stratified analysis of subjects whose amount of change in VAS for feeling of fatigue between before and after implementation of verbal fluency test and Stroop test was larger than 20 mm

The two tests requiring attention, i.e., verbal fluency test and Stroop test, impose a mental burden on the subjects, but the degree of the mental burden greatly varies from subject to subject. So, by reference to the report in which the subjects whose VAS value for the feeling of fatigue was larger than 20 mm are defined as “high-fatigue group” (Rat, A. C. et. al., Factors associated with fatigue in early arthritis: results from a multicenter national French cohort study., Arthritis Care Res., 64 (7), 1061-9 (2012), the subjects whose amount of change in VAS for the feeling of fatigue between before and after the implementation of the verbal fluency test and the Stroop test, before the initiation of ingestion of the test food, was larger than 20 mm were extracted as “subjects who were prone to be fatigued by intellectual work requiring attention and concentration,” and subjected to stratified analysis.

(ii) Stratified analysis of subjects whose score on “Fatigue-Inertia” scale in POMS2 inspection was 50 or higher

The scores in the POMS2 inspection are set so that a generation mean is 50. Thus, the subjects whose score on the “Fatigue-Inertia” scale was 50 or higher were extracted as “subjects who were highly fatigued in daily life” and subjected to stratified analysis.

### (6) Results

A. Overall analysis
(i) Verbal fluency test

The results were as shown in Table 3.

TABLE 3
Result of overall analysis of verbal fluency test

| | | | Before | Week 6 after | Week 12 after |
|---|---|---|---|---|---|
| Verbal fluency | | | initiation of | initiation of | initiation of |
| test | | Group | ingestion | ingestion | ingestion |
| Word starting | Measured | Control food | 12.1 ± 3.3 | 13.1 ± 3.6* | 13.7 ± 3.8** |
| with ‘a’ | value | group | | | |
| | | Test food | 11.4 ± 4.1 | 13.2 ± 3.9** | 14.1 ± 4.1** |
| | | group | | | |
| | Amount of | Control food | | 1.0 ± 2.8 | 1.7 ± 3.1 |
| | change from | group | | | |
| | before | Test food | | 1.9 ± 3.3 | 2.8 ± 3.1 |
| | initiation of | group | | | |
| | ingestion | | | | |
| Animal name | Measured | Control food | 19.5 ± 4.4 | 19.6 ± 4.7 | 20.9 ± 3.9* |
| | value | group | | | |
| | | Test food | 18.3 ± 4.1 | 19.4 ± 4.0* | 20.1 ± 4.6** |
| | | group | | | |
| | Amount of | Control food | | 0.2 ± 3.7 | 1.4 ± 3.9 |
| | change from | group | | | |
| | before | Test food | | 1.2 ± 3.2 | 1.9 ± 3.9 |
| | initiation of | group | | | |
| | ingestion | | | | |

*p < 0.05
**p < 0.01 (intra-group comparison with before initiation of ingestion)

No significant inter-group difference was observed between the test food group and the control food group, in either the actually measured value or the amount of change from before the initiation of ingestion (hereinafter sometimes referred to simply as “amount of change”) in the verbal fluency test. However, for the “words starting with ‘a,’” the test food group showed a larger amount of change (amount of increase) at Week 12 after the initiation of ingestion than that of the control food group, which amount of change was observed to be marginally significant as compared with that of the control food group (p=0.094). For the “animal names,” the actually measured value at Week 6 after the initiation of ingestion was significantly increased from that before the initiation of ingestion only in the test food group (p<0.05). The above results suggested the possibility that the ingestion of the decomposition product of whey may improve the judgement function (executive function).

(ii) Stroop test

The results were as shown in Table 4.

TABLE 4
Result of overall analysis of Stroop test

| | | | Before | Week 6 after | Week 12 after |
|---|---|---|---|---|---|
| | | | initiation of | initiation of | initiation of |
| Stroop test | | Group | ingestion | ingestion | ingestion |
| Character | Measured | Control food | 0.0 ± 0.1 | 0.2 ± 0.9 | 0.1 ± 0.3 |
| meaning | value | group | | | |
| (number of | | Test food | 0.1 ± 0.3 | 0.0 ± 0.2 | 0.1 ± 0.3 |
| errors) | | group | | | |
| | Amount of | Control food | | 0.1 ± 0.9 | 0.1 ± 0.3 |
| | change from | group | | | |
| | before | Test food | | 0.0 ± 0.4 | 0.0 ± 0.5 |
| | initiation of | group | | | |
| | ingestion | | | | |
| Character | Measured | Control food | 0.1 ± 0.3 | 0.1 ± 0.4 | 0.2 ± 0.7 |
| color | value | group | | | |
| (number of | | Test food | 0.2 ± 0.5 | 0.2 ± 0.4 | 0.2 ± 0.6 |
| errors) | | group | | | |
| | Amount of | Control food | | 0.0 ± 0.4 | 0.1 ± 0.7 |
| | change from | group | | | |
| | before | Test food | | 0.0 ± 0.6 | 0.0 ± 0.6 |
| | initiation of | group | | | |
| | ingestion | | | | |

No significant inter-group difference was observed between the test food group and the control food group in the Stroop test. However, in the test in which the subjects were made to read aloud the “meanings of words composed of characters,” the actually measured value of the test food group at Week 6 after the initiation of ingestion was smaller than that of the control food group, and the amounts of change (amounts of increase) of the test food group at Week 6 after the initiation of ingestion and at Week 12 after the initiation of ingestion were smaller than those of the control food group. The above results suggested the possibility that the ingestion of the decomposition product of whey may improve the attention function and judgement function (executive function).

### B. Stratified Analysis

(i) Stratified analysis of subjects whose amount of change in VAS for feeling of fatigue was larger than 20 mm

For the amounts of changes in the verbal fluency test and the Stroop test, the subjects whose amount of change in VAS for the feeling of fatigue between before and after the implementation of the verbal fluency test and the Stroop test was larger than 20 mm were subjected to stratified analysis. The number of subjects as the target for analysis was 19 (men: 6 and women: 13) for the test food group and 21 (men: 6 and women: 15) for the control test group.

The results were as shown in Table 5.

TABLE 5
Results (1) of stratified analysis of verbal fluency test and Stroop test

| | | | Week 6 after | Week 12 after |
|---|---|---|---|---|
| | | | initiation of | initiation of |
| Test | | Group | ingestion | ingestion |
| Verbal fluency | | | | |
| test | | | | |
| Word starting | Amount of | Control food | 1.0 ± 3.7 | 1.3 ± 3.6 |
| with ‘a’ | change from | group | | |
| | before initiation | Test food group | 3.3 ± 2.5**,# | 2.6 ± 3.0** |
| | of ingestion | | | |
| Animal name | Amount of | Control food | −0.6 ± 4.2 | 0.5 ± 3.7 |
| | change from | group | | |
| | before initiation | Test food group | 1.3 ± 2.6* | 0.8 ± 4.5 |
| | of ingestion | | | |
| Stroop test | | | | |
| Character | Amount of | Control food | 0.1 ± 0.3 | 0.0 ± 0.2 |
| meaning | change from | group | | |
| (number of | before initiation | Test food group | −0.1 ± 0.3# | −0.1 ± 0.3 |
| errors) | of ingestion | | | |
| Character | Amount of | Control food | 0.1 ± 0.4 | 0.3 ± 1.0 |
| color | change from | group | | |
| (number of | before initiation | Test food group | −0.1 ± 0.2 | 0.0 ± 0.5 |
| errors) | of ingestion | | | |

*p < 0.05
**p < 0.01 (intra-group comparison with before initiation of ingestion)
#p < 0.05 (inter-group comparison at each time point)

For the “words starting with ‘a’” in the verbal fluency test, the test food group showed a significantly large amount of change at Week 6 after the initiation of ingestion as compared with the control food group (p<0.05). Only in the test food group, the actually measured values at Week 6 after the initiation of ingestion and at Week 12 after the initiation of ingestion significantly increased from the value before the initiation of ingestion (p<0.01). For the “animal names” in the verbal fluency test, the actually measured values at Week 6 after the initiation of ingestion significantly increased from the value before the initiation of ingestion (p<0.05), only in the test food group. In the test in which the “meanings of words composed of characters” were read aloud in the Stroop test, the test food group showed a significantly large amount of change (amount of decrease) at Week 6 after the initiation of ingestion as compared with the control food group (p<0.05). The above results verified that the ingestion of the decomposition product of whey improves the attention function and judgement function (executive function) of the “subjects who are prone to be fatigued by intellectual work requiring attention and concentration.”

(ii) Stratified analysis of subjects whose score on “Fatigue-Inertia” scale in POMS2 was 50 or higher

For the amounts of changes in the verbal fluency test and the Stroop test, the subjects whose score on the “Fatigue-Inertia” scale in POMS2 was 50 or higher were subjected to stratified analysis. The number of subjects as the target for analysis was 12 (men: 4 and women: 8) for the test food group and 13 (men: 4 and women: 9) for the control test group.

The results were as shown in Table 6.

TABLE 6
Results (2) of stratified analysis of verbal fluency test and Stroop test

| | | | Week 6 after | Week 12 after |
|---|---|---|---|---|
| | | | initiation of | initiation of |
| Test | | Group | ingestion | ingestion |
| Verbal fluency | | | | |
| test | | | | |
| Word starting | Amount of | Control food | 0.8 ± 2.4 | 2.7 ± 3.1** |
| with ‘a’ | change from | group | | |
| | before initiation | Test food group | 3.1 ± 2.9**,# | 4.1 ± 3.7** |
| | of ingestion | | | |
| Animal name | Amount of | Control food | −1.2 ± 3.5 | 1.1 ± 3.5 |
| | change from | group | | |
| | before initiation | Test food group | 1.2 ± 2.7 | 2.3 ± 4.6 |
| | of ingestion | | | |
| Stroop test | | | | |
| Character | Amount of | Control food | 0.5 ± 1.7 | 0.0 ± 0.0 |
| meaning | change from | group | | |
| (number of | before initiation | Test food group | −0.2 ± 0.4 | −0.1 ± 0.5 |
| errors) | of ingestion | | | |
| Character | Amount of | Control food | 0.0 ± 0.6 | −0.1 ± 0.5 |
| color | change from | group | | |
| (number of | before initiation | Test food group | 0.0 ± 0.0 | 0.3 ± 0.9 |
| errors) | of ingestion | | | |

*p < 0.05
**p < 0.01 (intra-group comparison with before initiation of ingestion)
#p < 0.05 (inter-group comparison at each time point)

For the “words starting with ‘a’” in the verbal fluency test, the test food group showed a significantly large amount of change (amount of increase) at Week 6 after the initiation of ingestion as compared with the control food group (p<0.05). Only in the test food group, the actually measured value at Week 6 after the initiation of ingestion significantly increased from the value before the initiation of ingestion (p<0.01). For the “animal names” in the verbal fluency test, a tendency was observed that the amount of change (amount of increase) of the test food group at Week 6 after the initiation of ingestion was larger than that of the control food group (p=0.072). In the Stroop test, no significant inter-group difference was observed between the test food group and the control food group, but in the test in which the “meanings of words composed of characters” were read aloud, the test food group showed a larger amount of change (amount of decrease) at Week 6 after the initiation of ingestion than that of the control food group. The above results verified that the ingestion of the decomposition product of whey improves the attention function and judgement function (executive function) of the “subjects who are highly fatigued in daily life.”

From the above results, it was concluded that, since the ingestion of the tablets containing the decomposition product of whey improved the scores of the verbal fluency test and the Stroop test of the subjects who were aware of their own frequent forgetfulness and many mistakes, the decomposition product of whey improves the attention function and judgement function (executive function). Further, the results of the stratified analysis using the VAS inspection for the feeling of fatigue and the POMS2 inspection demonstrated that the ingestion of the decomposition product of whey more prominently improves the attention function and judgement function of the subjects who are prone to be fatigued by intellectual work requiring attention and the subjects who are highly fatigued in daily life.

1-8. (canceled)

9. A method for improving, maintaining or ameliorating an attention function and/or a judgement function, comprising feeding or administering an effective amount of an enzymatic decomposition product of milk protein or a composition comprising the enzymatic decomposition product of milk protein to a subject in need thereof.

10. (canceled)

11. The method according to claim 9, wherein the enzymatic decomposition product of milk protein is an enzymatic decomposition product of whey protein.

12. The method according to claim 9, wherein the enzymatic decomposition product of milk protein comprises a peptide having an amino acid sequence of GTWY (SEQ ID NO: 1) and a peptide having an amino acid sequence of WY.

13. The method according to claim 9, wherein the subject is prone to be fatigued by intellectual work.

14. The method according to claim 13, wherein the intellectual work is work which requires attention and/or concentration.

15. The method according to claim 9, wherein the subject is fatigued or prone to be fatigued in daily life.

16. The method according to claim 9, wherein the daily administering amount of the enzymatic decomposition product ranges 0.01 to 100 g (in terms of solid content) for a human.

17. The method according to claim 9, wherein the enzymatic decomposition product or the composition is in the form of a food.
